(19) United States
(12) Patent Application Publication
(10) Pub. No.: US 2008/0260863 A1
(43) Pub. Date: Oct. 23, 2008

(54) COMPOSITIONS FOR MUCOCILIARY CLEARANCE AND METHODS FOR ADMINISTERING SAME

(75) Inventors: W. Randolph WARNER, Punta Gorda, FL (US); Werner Gutmann, Powhatan, VA (US)

Correspondence Address: MCGUIREWOODS, LLP, 1750 TYSONS BLVD, SUITE 1800, MCLEAN, VA, 22102, US

(73) Assignee: PRE Holding, Inc., Midlothian, VA (US)

(21) Appl. No.: 12/106,831

(22) Filed: Apr. 21, 2008

Related U.S. Application Data
(60) Provisional application No. 60/913,160, filed on Apr. 20, 2007.

(51) Int. Cl. A61K 33/14

(52) U.S. Cl. 424/677

(57) ABSTRACT

Sterile, preservative-free premeasured hypertonic saline solutions may be administered to individuals afflicted with cystic fibrosis to increase mucolciliary clearance and to improve lung function. In particular, a 7% hypertonic saline solution may be administered at least 2 times a day, using a jet and/or vibrating-mesh nebulizer to an individual afflicted with cystic fibrosis.

### CROSS-REFERENCE TO RELATED APPLICATIONS

This application claims priority under 35 U.S.C. § 119(e) to provisional U.S. Patent Application No. 60/913,160, filed on Apr. 20, 2007, the disclosure of which is expressly incorporated by reference herein in its entirety.

### BACKGROUND

1. Field of the Invention

The invention relates to compositions and methods for administering sterile, preservative-free pre-measured hypertonic saline solutions to individuals afflicted with cystic fibrosis. In particular, the invention relates to administering at least 2 times a day, a hypertonic saline solution using a jet and/or vibrating-mesh nebulizer to an individual afflicted with CF. Moreover, the invention relates to blow-filled vials and kits for providing sterile, preservative-free premeasured hypertonic saline solutions having a concentration in the range of about 3.5% to about 9%.

2. Related Art

Cystic fibrosis (CF), also called mucoviscidosis, is an inherited disease of the exocrine glands affecting primarily the GI and respiratory systems. 1 in 2500 children are born with CF, and it is one of the most common fatal inherited diseases. It is most prevalent among Europeans and Ashkenazi Jews. In fact, one in twenty-two people of European descent carry the gene for CF, making it the most common genetic disease among them.

CF is carried as an autosomal recessive trait. The responsible gene has been localized on the long arm of chromosome 7q. The gene encodes for a protein called the cystic fibrosis transmembrane conductance regulator (CFTR). The CFTR protein is responsible for creating sweat, digestive juices, and mucous.

The most common symptoms of CF include difficulty breathing and insufficient enzyme production in the pancreas. Moreover, thick mucous production, as well as a low immune system, results in frequent lung infections, which may be treated by oral and intravenous antibiotics and other medications. There are several other symptoms of CF, such as sinus infections, poor growth, diarrhea, and infertility.

The lungs of patient afflicted with CF are generally normal at birth. Pulmonary damage is most likely initiated from diffuse obstruction in the small airways by abnormally thick mucus secretions. Bronchiolitis and mucopurulent plugging of the airways occur secondary to obstruction and infection. The lung disease in cystic fibrosis is characterized by impaired mucociliary clearance. Hypertonic saline (HS) has been shown to enhance mucociliary clearance in-vitro, and this may act to lessen the destructive inflammatory process in the airways.

Inhaled hypertonic saline, and in particular, a 7% hypertonic saline solution, may acutely increase mucociliary clearance and may improve lung function in individuals afflicted with CF. The hypertonic saline solution acts as a solvent to facilitate removal of mucous and other debris from the nasal passages. The high salt concentration draws fluid out of and shrinks the membranes, thus reducing decongestion and improving air flow. Moreover, inhaled hypertonic saline therapy may be associated with reductions in the number of exacerbations, antibiotic use for exacerbations, absenteeism from school or work, and the inability to engage in other, usual activities.

Currently, 7% hypertonic saline solutions for inhalation are not commercially available. Compared with commercially prepared saline solutions, compounded hypertonic saline solution has a relatively short shelf life, so the CF Services Pharmacy has not decided to offer a compounded version product. Instead, the CF Foundation and the CF Services Pharmacy recommend preparing a 7% hypertonic saline solution for inhalation by mixing commercially available 0.9% and 10% solutions at the time of use. Moreover, patients are preparing hypertonic saline solutions by following recipes or guidelines, found via the internet and other, sources using tap water, pickling and/or canning salt, and commercially available nasal bulbs.

There several disadvantages associated with this configuration and administration practice. Parents, care givers, and others typically do not have adequate experience diluting or preparing this medication, resulting in contamination or inappropriate dosing, among other problems. Therefore, there is a need for an improved hypertonic saline inhalation solution, system, kit, and method for the treatment of mucociliary clearance with individuals afflicted with CF.

### SUMMARY OF THE INVENTION

The invention satisfies the above needs by providing sterile, preservative-free premeasured hypertonic saline solutions suitable for inhalation therapy in individuals afflicted with CF. Moreover, the invention provides comprehensive kit and methods for caregivers to administer hypertonic saline therapy to a patient in need.

Accordingly, in one aspect of the invention, a hypertonic saline therapy is provided for administration to a patient afflicted with a condition. The therapy may be a sterile, preservative-free, non-pyrogenic hypertonic saline solution stored in an ampoule and having a concentration in the range from about 3.5% to about 9%.

Further, the solution may have a concentration of about 7%. The patient may be afflicted with cystic fibrosis. The therapy may be administered with a vibrating mesh nebulizer or a jet nebulizer. The therapy may be a 7% solution and may be administered twice daily. The ampoule may contain about 4 mL of saline solution. The hypertonic solution may also include an additional pharmacologically active agent, which may include an anti-infective, an anti-mucolytic, a buffer, or an anti-inflammatory.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawing, which is included to provide a further understanding of the invention, is incorporated in and constitutes a part of this specification; illustrate embodiments of the invention and together with the detailed description serve to explain the principles of the invention. No attempt is made to show structural details of the invention in more detail than may be necessary for a fundamental understanding of the invention and various ways in which it may be practiced.

FIG. 1 is a graph showing percentage change in FEV₁ from baseline to 15 minutes after bronchodilator and 15 minutes after the initial dose of hypertonic saline. Data points represent means and error bars represent standard deviations.

FIG. 2 is a chart showing severity of cough as measured by the VAS during the tolerance test.

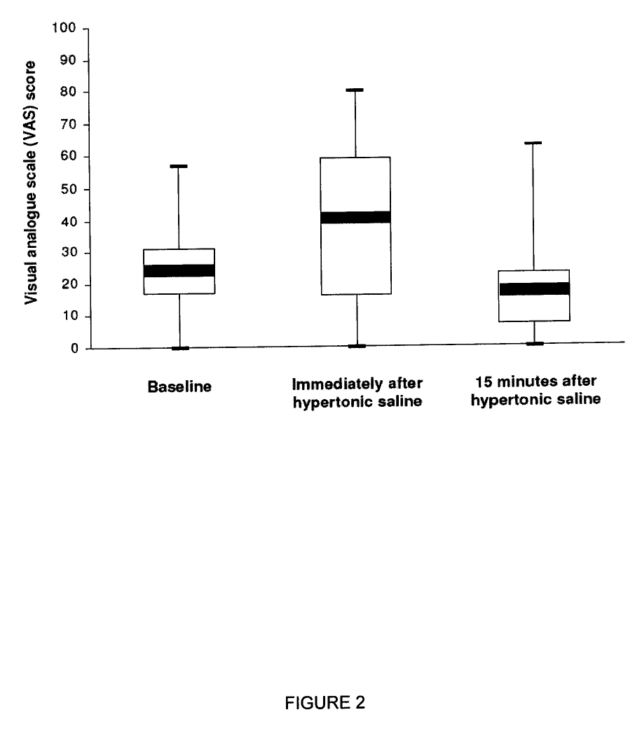

FIG. 3 is a chart showing severity of dyspnoea as measured by the VAS during the tolerance test.

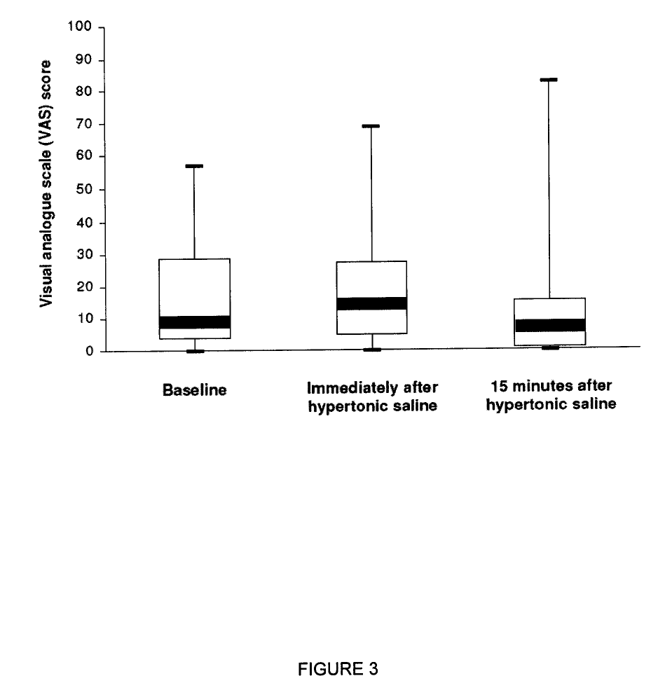

FIG. 4 is a chart showing severity of wheeze as measured by the VAS during the tolerance test.

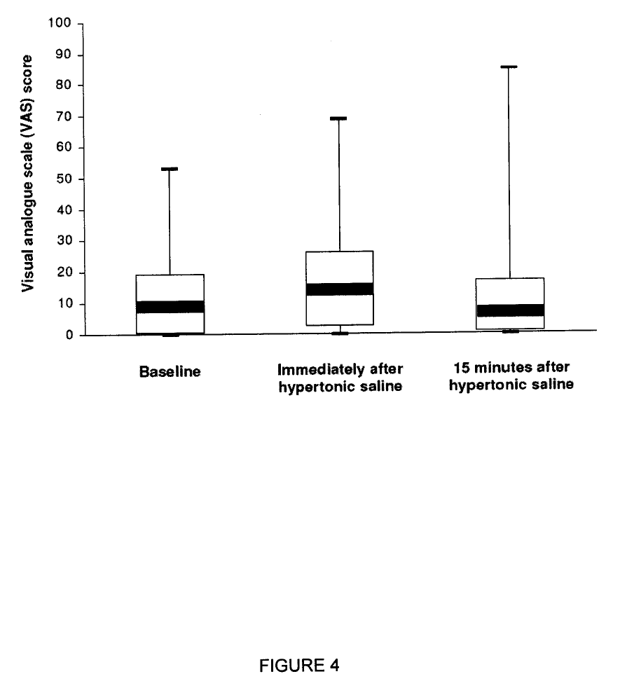

FIG. 5 is a chart showing severity of chest tightness as measured by the VAS during the tolerance test.

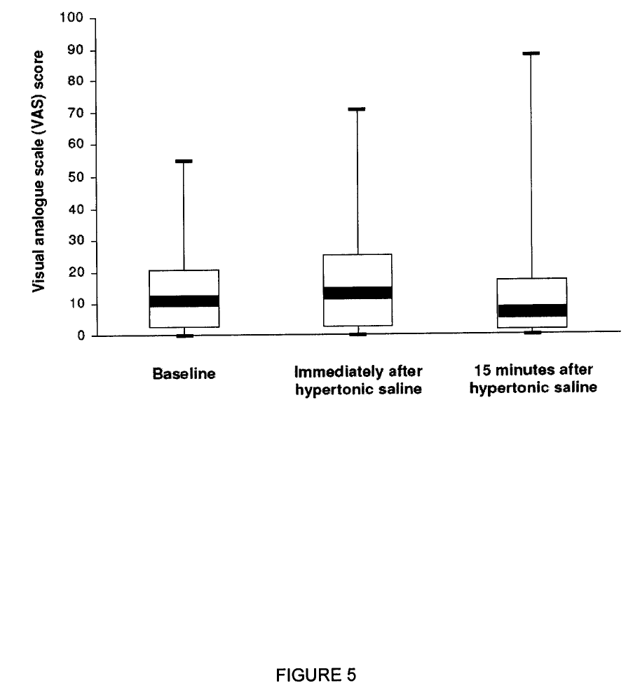

FIG. 6 is a chart showing density of P. aeruginosa in sputum at Day 0 and Day 14 in each group. CFU/g: colony forming units per gram of sputum.

FIG. 7 is a chart showing density of S. aureus in sputum at Day 0 and Day 14 in each group. CFU/g: colony forming units per gram of sputum.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a construct” is a reference to one or more constructs and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, osmolality, temperature, pressure, time and the like, is, for example, from 1 to 90, specifically from 20 to 80, more specifically from 30 to 70, it is intended that values such as 15 to 85, 22 to 68, 43 to 51, 30 to 32 etc., are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

### DEFINITIONS

The terms “active agent,” “drug,” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect.

By “pharmaceutically acceptable carrier” is meant a material or materials that are suitable for drug administration and not biologically or otherwise undesirable, i.e., that may be administered to an individual along with an active agent without causing any undesirable biological effects or interacting in a deleterious manner with any of the other components of the pharmaceutical formulation in which it is contained.

Similarly, a “pharmacologically acceptable” salt, ester or other derivative of an active agent as provided herein is a salt, ester or other derivative that is not biologically or otherwise undesirable.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” individuals with CF, as the term “treating” is used herein, encompasses treatment of mucous accumulation in a clinically symptomatic individual.

The terms “condition,” “disease” and “disorder” are used interchangeably herein as referring to a physiological state that can be prevented or treated by administration of a pharmaceutical formulation as described herein. Exemplary diseases and conditions may include, but not limited to, asthma, acute lower respiratory tract infection, pneumonia, infant respiratory distress syndrome, croup, bronchitis, and pertussis.

The term “patient” as in treatment of “a patient” refers to a mammalian individual afflicted with or prone to a condition, disease or disorder as specified herein, and includes both humans and animals.

An “aerosol,” as used herein, is a system comprising a continuous gas phase and, dispersed therein, a discontinuous phase of liquid and/or solid particles.

“Nebulization,” as used herein, refers to the conversion of a liquid, such as a liquid solution, emulsion, or suspension, into an aerosol. Thus, a nebulized aerosol comprises liquid droplets dispersed in a continuous gas phase. The liquid droplet may optionally comprise solid particles which are suspended within the droplets.

A “nebulizer,” as used herein, is a device which is capable of converting a liquid material into a nebulized aerosol which is typically inhalable by a human via the nose or the mouth.

The term “ampoule” as used herein generally refers to a container for aseptic or sterile compositions in the pharmaceutical and biological industries. Product information such as batch number and expiration date may be printed on the ampoule directly, or on a paper or plastic label which is then affixed to the body of the ampoule surface. Additionally, the term “ampoule” as used herein may generally refer to a blow-fill seal vial.

The “residual volume” of a nebulizer is the volume of liquid which cannot be aerosolized, but which remains in the reservoir of the device.

The invention relates to hypertonic saline therapy for patients afflicted with an upper respiratory condition. In one embodiment, the inhalation solution of the invention may be provided in sterile, unit dose treatments, thereby eliminating the need for preservatives in the solution. Another embodiment provides a sterile, premixed, premeasured inhalation solution in concentrations in the range of about 3.5% to about 9%. More specifically, the hypertonic saline solutions may be available in concentrations of about 3.5%, about 6%, and about 7%. The sterile, premixed, premeasured hypertonic saline solutions may reduce the possibility of introducing contaminants into the patient when administered. Thus, the risk of an opportunistic infection in the patient is reduced. Additionally, providing the inhalation solution in such a manner eliminates the need to dilute the conventionally available hypertonic saline solutions to obtain proper dosages for treatment.

According to one embodiment, the inhalation solution of the invention may be packaged in blow-filled ampoules. The blow-filled ampoules may be prefilled with hypertonic saline solutions having concentrations of about 3.5%, about 6%, about 7%, and about 9%. Moreover, the blow-filled ampoules may be prefilled with about 4 ml of hypertonic saline solution. Moreover, the inhalation solution packaged in the blow-filled ampoule may be preservative-free. The ampoules of the invention may be made from glass, plastic, or any other suitable material known by those of skill in the art. Specifically, the ampoules may be plastic blow-fill seal vial.

In one embodiment, the inhalation solution of the invention may be administered by nebulizer such as jet nebulizer, ultrasonic nebulizer, or breath-enhanced nebulizer. In a specific embodiment, the inhalation solution may be administered in a jet nebulizer.

The hypertonic saline solution may be adapted for pulmonary administration by oral or nasal administration. This means that it fulfills the standard requirements for formulations intended for these routes of administration as found in the current pharmacopeias or guidance documents issued by regulatory bodies such as the U.S. Food and Drug Administration (FDA).

There are two widely known classes of medical nebulizers: the jet nebulizer, which is powered by compressed air, and the ultrasonic nebulizer, which derives the energy required to aerosolize drugs from high frequency sound waves. Jet nebulizers are driven either by a portable compressor or from a central air supply.

Jet nebulizers that meet the criteria for carrying out the invention include, for example, high-performance devices such as the PARI LC or PARI LC SPRINT, driven by an appropriate compressor such as the PARI Boy N (PRONEB ultra in the USA). In particular, a nebulizer that carries out the invention may also achieve a very high FPF, such as an electronic perforated vibrating membrane nebulizer, and may include member of the PARI eFlow series. Other optional nebulizers may include ultrasonic nebulizers, electrohydrodynamic nebulizers, non-vibrating or non-perforated membrane nebulizers, or nebulizers combining two or more of these types.

Essentially, in a medical nebulaizer, a high-speed air flow through a narrow, nozzle orifice entrains and disperses the liquid into droplets (primary generation) via a viscosity-induced instability. Droplet dispersion is improved by impaction on a baffle structure adjacent to the nozzle orifice transferring kinetic energy further into increased droplet surface area (secondary generation). The resulting droplet size distribution still contains only a small fraction of respirable aerosol (droplets in the range of about 5 to about 6 μm in size) and the large droplets are recirculated within the nebulizer by means of secondary impaction structures. This process is associated with evaporation effects that cause the gas phase to be nearly saturated with vapor, as well as a temperature decrease within the nebulizer.

The hypertonic saline solutions of the invention may be modified to accommodate the particular concentration of hypertonic saline solution used and the particular patient population the hypertonic saline solution is administered to. Formulation characteristics that may be modified include, for example, additional pharmacological active agents, pH, and osmolality. Additionally, polymeric excipients may be useful in the liquid formulations of the invention, for among other things, to obtain slow release profile of the drug. These polymeric excipients include for example, chitosan, hydroxypropylmethylcellulose, dextrans, and kollidon.

According to another embodiment, the hypertonic saline solution of the invention may have a pH in the range of about pH 4 to about pH 8, in particular from about pH 5 to pH 7.5. The pH may be adjusted by the addition of a solution containing an acidic salt or an acid (e.g., hydrochloric or sulfuric acid); or of a basic salt or a base (e.g., sodium hydroxide). In case of insufficient stability of the formulation, aqueous buffered systems (citrate, acetate, phosphate) may be added to keep the pH within a physiologically acceptable range. A variety of buffers known in the art may be used in the formulation of the invention, such as various salts of organic or inorganic acids, bases, or amino acids, and including various forms of citrate, phosphate, tartrate, succinate, adipate, maleate, lactate, acetate, bicarbonate, or carbonate ions.

In another embodiment of the invention, other pharmacologically active agents may be combined with the hypertonic saline solution of the invention, such as anti-infective agents, anti-mucolytics, and anti-inflammatory agents. Examples of anti-infective agents, whose class or therapeutic category is herein understood as comprising compounds which are effective against bacterial, fungal, and viral infections, i.e. encompassing the classes of antimicrobials, antibiotics, antifungals, antiseptics, and antivirals, that may be used in combination with the saline therapy of the invention may include penicillins, including benzylpenicillins (penicillin-G-sodium, clemizone penicillin, benzathine penicillin G), phenoxypenicillins (penicillin V, propicillin), aminobenzylpenicillins (ampicillin, amoxycillin, bacampicillin), acylaminopenicillins (azlocillin, mezlocillin, piperacillin, apalcillin), carboxypenicillins (carbenicillin, ticarcillin, temocillin), isoxazolyl penicillins (oxacillin, cloxacillin, dicloxacillin, flucloxacillin), and amiidine penicillins (mecillinam); cephalosporins, including cefazolins (cefazolin, cefazedone); cefuroximes (cerufoxim, cefamdole, cefotiam), cefoxitins (cefoxitin, cefotetan, latamoxef, flomoxef), cefotaximes (cefotaxime, ceftriaxone, ceftizoxime, cefinenoxime), ceftazidimes (ceftazidime, cefpirome, cefepime), cefalexins (cefalexin, cefaclor, cefadroxil, cefradine, loracarbef, cefprozil), and cefiximes (cefixime, cefpodoxim proxetile, cefuroxime axetil, cefetamet pivoxil, cefotiam hexetil), loracarbef, cefepim, clavulanic acid/amoxicillin, Ceftobiprole; synergists, including beta-lactamase inhibitors, such as clavulanic acid, sulbactam, and tazobactam; carbapenems, including imipenem, cilastin, meropenem, doripenem, tebipenem, ertapenem, ritipenam, and biapenem; monobactams, including aztreonam; aminoglycosides, such as apramycin, gentamicin, amikacin, isepamicin, arbekacin, tobramycin, netilmicin, spectinomycin, streptomycin, capreomycin, neomycin, paromoycin, and kanamycin; macrolides, including erythromycin, clarythromycin, roxithromycin, azithromycin, dithromycin, josamycin, spiramycin and telithromycin; gyrase inhibitors or fluroquinolones, including ciprofloxacin, gatifloxacin, norfloxacin, ofloxacin, levofloxacin, perfloxacin, lomefloxacin, fleroxacin, garenoxacin, clinafloxacin, sitafloxacin, prulifloxacin, olamufloxacin, caderofloxacin, gemifloxacin, balofloxacin, trovafloxacin, and moxifloxacin; tetracyclins, including tetracyclin, oxytetracyclin, rolitetracyclin, minocyclin, doxycycline, tigecycline and aminocycline; glycopeptides, inlcuding vancomycin, teicoplanin, ristocetin, avoparcin, oritavancin, ramoplanin, and peptide 4; polypeptides, including plectasin, dalbavancin, daptomycin, oritavancin, ramoplanin, dalbavancin, telavancin, bacitracin, tyrothricin, neomycin, kanamycin, mupirocin, paromomycin, polymyxin B and colistin; sulfonamides, including sulfadiazine, sulfamethoxazole, sulfalene, co-trimoxazole, co-trimetrol, co-trimoxazine, and co-tetraxazine; azoles, including clotrimazole, oxiconazole, miconazole, ketoconazole, itraconazole, fluconazole, metronidazole, tinidazole, bifonazol, ravuconazol, posaconazol, voriconazole, and omidazole and other antifungals including flucytosin, griseofluvin, tonoftal, naftifin, terbinafin, amorolfin, ciclopiroxolamin, echinocandins, such as micafingin, caspofungin, anidulafungin; nitrofurans, including nitrofurantoin and nitrofuranzone;—polyenes, including amphotericin B, natamycin, nystatin, flucocytosine; other antibiotics, including tithromycin, lincomycin, clindamycin, oxazolindiones (linzezolids), ranbezolid, streptogramine A+B, pristinamycin aA+B, Virginiamycin A+B, dalfopristin/qiunupristin (Synercid), chloramphenicol, ethambutol, pyrazinamid, terizidon, dapson, prothionamid, fosfomycin, fucidinic acid, rifampicin, isoniazid, cycloserine, terizidone, ansamycin, lysostaphin, iclaprim, mirocin B17, clerocidin, filgrastim, and pentamidine; antivirals, including acyclovir, ganciclovir, birivudin, valaciclovir, zidovudine, didanosin, thiacytidin, stavudin, lamivudin, zalcitabin, ribavirin, nevirapirin, delaviridin, trifluridin, ritonavir, saquinavir, indinavir, foscamet, amantadin, podophyllotoxin, vidarabine, tromantadine, and proteinase inhibitors; plant extracts or ingredients, such as plant extracts from chamomile, hamamelis, echinacea, calendula, papain, pelargonium, essential oils, myrtol, pinen, limonen, cineole, thymol, mentol, alpha-hederin, bisabolol, lycopodin, vitapherole; wound healing compounds including dexpantenol, allantoin, vitamins, hyaluronic acid, alpha-antitrypsin, anorganic and organic zinc salts/compounds, interferons (alpha, beta, gamma), tumor necrosis factors, cytokines, interleukins.

Examples of potentially useful mucolytics that may be in combination with the saline therapy of the invention may be DNase, P2Y2-agonists (denufosol), heparinoids, guaifenesin, acetylcysteine, carbocysteine, ambroxol, bromhexine, lecithins, myrtol, and recombinant surfactant proteins.

Examples of potentially useful anti-inflammatory compounds that may be used in combination with the saline therapy of the invention may include glucocorticoids and non-steroidal anti-inflammatory agents such as betamethasone, beclomethasone, budesonide, ciclesonide, dexamethasone, desoxymethasone, fluoconolone acetonide, flucinonide, flunisolide, fluticasone, icomethasone, rofleponide, triamcinolone acetonide, fluocortin butyl, hydrocortisone, hydroxycortisone-17-butyrate, prednicarbate, 6-methylprednisolone aceponate, mometasone furoate, elastane, prostaglandin, leukotriene, bradykinin antagonists, non-steroidal anti-inflammatory drugs (NSAIDs), such as ibuprofen, indometacin, including any pharmaceutically acceptable salts, esters, isomers, stereoisomers, diastereomers, epimers, solvates or other hydrates, prodrugs, derivatives, or any other chemical or physical forms of active compounds comprising the respective active moieties.

Additional constituent elements of the liquid formulations of the invention may include a buffer, a pH-adjusting agent, a surfactant or anti-adsorbant, a wetting agent, a gelling agent, a drying agent, an osmolality adjusting agent, or virtually any other additive or carrier, depending upon the desired dosage form.

Buffers may be useful in the invention for, among other purposes, manipulation of the total pH of the liquid formulation. A variety of buffers known in the art may be used in the formulation of the invention, such as various salts of organic or inorganic acids, bases, or amino acids, and including various forms of citrate, phosphate, tartrate, succinate, adipate, maleate, lactate, acetate, bicarbonate, or carbonate ions. The pH of the formulation changes according to the amount of buffer used. Depending upon the dosage form it may alternatively be advantageous to use buffers in different concentrations or to use other additives to adjust the pH of the formulation to encompass other ranges. Useful pH ranges for formulations of the invention include a pH of about 4.0 to a pH of about 7.0.

It may also be advantageous to employ surfactants in the liquid formulations of the invention. Surfactants or anti-adsorbants that prove useful include polyoxyethylenesorbitans, polyoxyethylenesorbitan monolaurate, polysorbate-20, such as Tween-20™, polysorbate-80, hydroxycellulose, and genapol, vitamin E-TPGS and lecithins or lecithin constituents.

Additional useful additives are readily determined by those of skill in the art, according to particular needs or intended uses of the compositions and formulator. In particular, osmolalities for administration of the liquid formulations of the invention may be in the range of about 200 to about 400 mOsm/kg.

The description and examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the pharmaceutical sciences or related fields are intended to be within the scope of the appended claims.

The disclosures of all references and publications cited above are expressly incorporated by reference in their entireties to the same extent as if each were incorporated by reference individually.

### EXAMPLES

### Specific Example 1

### Tolerability of Hypertonic Saline Delivered by a High-Output Nebulizer

This example provides a study to determine whether inhaling hypertonic saline via the eFlow nebulizer either twice daily or four times daily was tolerable for 85% of people with CF. Furthermore, this example illustrates the effects of the two regimens on lung function, oxygenation, symptoms, quality of life, bacterial load, and adverse events.

A 2-week, randomized, parallel-group study compared twice-daily with four-times-daily inhalation of about 4 mL nebulized about 6% hypertonic saline. This study used a concealed method of random allocation and data were analyzed according to the intention-to-treat principle. All subjects, their clinicians, and the Study Coordinator remained blinded to treatment group allocation throughout the study.

The subjects in the study were required to meet the following criteria to be eligible for the study: aged at least 18 years; a confirmed diagnosis of CF, based on (i) a sweat chloride greater than about 60 mmol/L by quantitative pilocarpine iontophoresis; or (ii) the identification of genetic mutations at the CF gene; no non-routine antibiotics for a period of about 14 days; FEV₁ within about 10% of the best recorded value for the past 6 months; and FEV₁ at least about 40% predicted for height, age and gender.

The hypertonic saline solution used in the study was about 6% hypertonic saline (HYPERSAL 6, Pharmacy Department, Royal Children's Hospital, Parkville, Australia) which is available under the Specialty Hospital Product Scheme of Australia. Subjects were instructed to inhale about 4 mL of the solution twice daily or four times daily as per randomization. Subjects collected the solution from the Pharmacy Department as they were leaving the hospital after their baseline visit to preserve blinding of study staff.

An eFlow Rapid nebulizer (Pari, Hamburg, Germany) was supplied to all subjects. The anticipated nebulization time was about 4 minutes per dose.

Subjects who passed the tolerance test were dispensed enough ampoules of their allocated saline solution for twice-daily or four-times-daily inhalation, as per randomization, for two weeks. Subjects were advised to space out their scheduled doses as much as possible during their usual waking hours. Subjects were instructed to inhale the premedication before each dose, regardless of whether they had demonstrated a fall in lung function following inhalation of hypetonic saline during the tolerance test.

Subjects who were regularly using a bronchodilator at enrolment were advised to use their current bronchodilator before every dose. Subjects that were not regularly using a bronchodilator at enrolment were supplied with enough of the metered-dose inhalers used in their successful tolerance test to allow the same premedication to be performed before every saline dose for the duration of the study. These subjects also retained the Volumatic (Allen & Hanburys) spacer device used during the tolerance test and requested to use it when inhaling all premedication.

If subjects found their allocated inhalation regimen intolerable, they were advised to cease the inhalations until the symptoms settled and then to recommence at half the frequency. Thus, subjects in the twice-daily group would drop to one dose per day, while those in the four-times-daily group would drop to twice-daily. This instruction allowed the Study Coordinator to remain blinded while advising subjects on this issue.

Subjects were advised to contact the Study Coordinator if they were unable to tolerate the allocated regimen or experienced adverse events. Subjects were also contacted at Day 7 to remind them of this and to record any changes in medications.

Standard methods were used for measurement of height, weight, oximetry and spirometry, as well as for the calculation of body mass index and predicted spirometric values.

In addition to the acute symptoms occurring during the tolerance test, a visual analogue scale was also used to measure the overall severity of common CF symptoms over a 24-hour period at baseline and at Day 14. The symptoms rated were cough, chest congestion, shortness of breath, difficulty clearing sputum, fatigue and sleep disturbance.

Baseline medication use was recorded for each subject at the outset of the study. All alterations to baseline medication use during the study were documented in a case report form. Subjects were able to continue on all their routine medications throughout the entire study period. Prescription of additional medications was at the discretion of the treating physician.

Adverse events were defined as any adverse change from the subject's baseline (pre-treatment) condition. This included any clinical or laboratory test value abnormality which occurred during the course of the study after treatment had been initiated, whether considered related to the allocated saline solution or not. Adherence was assessed by counting returned sachets of hypertonic saline.

It was determined that if greater than about 15% of subjects found a regimen intolerable after two weeks, the regimen was unlikely to be widely useful in clinical practice. The primary outcome was therefore whether the proportion of subjects for whom the allocated regimen was intolerable was less than about 15 percent in each group. As a secondary outcome, the rate of intolerance in the two groups was compared with an estimate of the difference between the two proportions and a confidence interval for the difference calculated by the Newcombe-Wilson method without continuity correction.

Outcomes of the tolerability test, that is, acute change in lung function and symptoms, using the first dose were presented for all subjects with descriptive statistics. Unpaired t-tests were used to compare the change from Day 0 to Day 14 between the two groups in the secondary outcomes: lung function, body mass index, 24-hour symptom severity, oxygenation and density of P. aeruginosa and S. aureus in sputum. Where data were not normally distributed, a Mann-Whitney U test was used. Descriptive statistics were used to report the within-group changes in these outcomes for each group. Changes in medication use, acquisitions/loss of organisms, and adverse events were compared between the two groups with the use of the chi-square test or, in cases in which subgroups were small, Fisher's exact test. No adjustments for multiplicity were made across secondary outcomes.

The required sample size was calculated to allow identification of whether greater than about 15% of subjects found their regimen intolerable after two weeks. Allowing for an attrition rate of 11% from an equivalent pilot study, 3 subjects would be expected to drop out of each arm for reasons other than poor tolerance. If a further 3 subjects of the remaining 17 in either group found their regimen intolerable, that would indicate that the regimen was not tolerated well enough to be useful in clinical practice.

Forty-nine patients were approached about participation in the study, of whom 40 gave consent and were eligible for the study. Results of randomization are shown below in Tables 1-3 that immediately follow.

TABLE 1
Demographic and clinical characteristics of the 40 subjects.

| | Twice Daily | Four Times Daily |
|---|---|---|
| Characteristic | (N = 20) | (N = 20) |
| Age (yr) | 28 ± 6 | 27 ± 8 |
| Gender (% female) | 55% | 45% |
| Body Mass Index (kg/m²) | 22 ± 3 | 21 ± 3 |
| FEV₁ (% predicted) | 58 ± 18 | 61 ± 20 |
| FVC (% predicted) | 78 ± 20 | 83 ± 20 |
| FEF₂₅₋₇₅ (% predicted) | 26 ± 16 | 29 ± 20 |

TABLE 2
Baseline treatment regimen of the 40 subjects

| | Twice Daily | Four Times Daily |
|---|---|---|
| Characteristic | (N = 20) | (N = 20) |
| Regular bronchodilator use (%) | 50 | 70 |
| Regular rhDNase use (%) | 55 | 60 |
| Regular antibiotic use (%) | 65 | 60 |
| Regular hypertonic saline use (%) | 30 | 10 |

TABLE 3
Microbiological characteristics of sputum of the 30 subjects able
to provide an adequate sputum sample for processing at baseline.

| | Twice Daily | Four Times Daily |
|---|---|---|
| Characteristic | (N = 15) | (N = 15) |
| P. aeruginosa in sputum (%) | 87 | 100 |
| S. aureus in sputum (%) | 27 | 20 |
| Density of P. aeruginosa (log₁₀CFU/g) | 19 ± 19 | 19 ± 18 |
| Density of S. aureus (log₁₀CFU/g) | 12 ± 10 | 12 ± 12 |

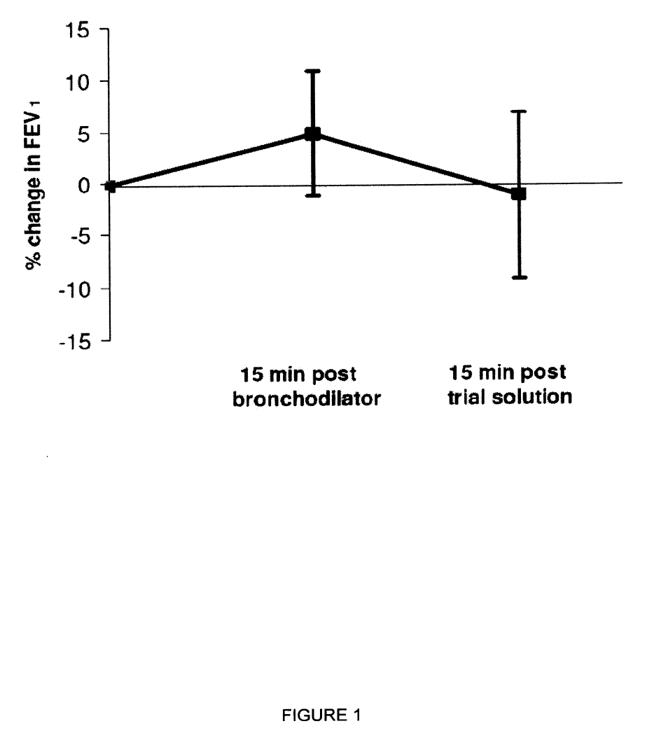

After the administration of a bronchodilator for tolerance testing, the mean improvement in the FEV₁ of all subjects was about 106 mL. After the subsequent inhalation of the first dose of hypertonic saline, the FEV₁ fell by a mean of about 131 mL (FIG. 1). Three subjects had a decrease in their FEV₁ of more than about 15 percent from baseline after the inhalation of hypertonic saline. These falls in FEV₁ were temporary, however, and by 15 minutes later all three subjects had a greater FEV₁ than at baseline. Thus all 40 subjects were eligible to continue in the study.

All four of the symptoms measured with the VAS after the inhalation of the first dose of hypertonic saline showed an increase in severity from baseline to the immediate post-saline measure. However, all median values had fallen to below baseline levels by 15 minutes later. No subjects found the symptoms of a single dose intolerable and all undertook to commence regular inhalations according to their randomly-allocated regimen. All were eligible to do so as the only subjects to have a greater than 15 percent fall in FEV₁ from baseline recovered within 15 minutes.

Results of two-week inhalation: None of the subjects in the group randomized to twice-daily inhalations were unable to tolerate the allocated regimen. Four (20%) of the 20 subjects allocated to four-times-daily inhalations were unable to tolerate their regimen. All four ceased inhalations (at Day 2, Day 4, Day 7 and Day 11) until their symptoms resolved, which took a mean (range) of 3 (0.5 to 3.5) days. All were able to recommence twice-daily dosing and continue this until the end of the two-week period.

Both groups showed within-group improvements in FEV₁ and FVC during the two-week treatment period (Table 4). The between group comparisons were not significant. The difference in improvement in FEV₁ in the four-times-daily group compared to the twice-daily group was 2 (95 percent confidence interval, 4 to 8) absolute percent. The difference in improvement in FVC in the four-times-daily group compared to the twice-daily group was 2 (95 percent confidence interval, −5 to 9) absolute percent.

TABLE 4
Within-group changes in lung function during two weeks
of regular hypertonic saline inhalation. Means (95
percent confidence intervals) are presented.

| | Twice Daily | Four Times Daily |
|---|---|---|
| Outcome | (N = 20) | (N = 20) |
| Percentage change in FEV₁ | 5 (2 to 8) | 7 (2 to 12) |
| from baseline | | |
| Percentage change in FVC | 6 (1 to 11) | 8 (3 to 13) |
| from baseline | | |

Severity of all symptoms tended to reduce for the group as a whole from Day 0 to Day 14, especially cough and chest congestion (Table 5). There were no significant between-group differences in symptom severity.

TABLE 5
Reductions in symptoms severity for all subjects. Means
(95 percent confidence intervals) are presented.

| | | Between-group |
|---|---|---|
| | Reduction from | difference in |
| | baseline among | change from |
| Symptom | all subjects (mm) | baseline * (mm) |
| Cough | 7 (1 to 13) | 5 (−7 to 17) |
| Chest congestion | 9 (3 to 15) | −4 (8 to −16) |
| Dyspnoea | 1 (−4 to 6) | −6 (3 to −15) |
| Difficulty clearing sputum | 5 (0 to 10) | −5 (6 to −16) |
| Fatigue | 4 (−3 to 11) | −10 (4 to −24) |
| Sleep disturbance | 3 (−2 to 8) | −5 (6 to −16) |

* Positive values represent greater reductions in symptom severity in the four-times-daily group.

The change in body mass index in the two groups during the two-week treatment period are shown in Table 6. The between-group comparisons were not significant. The 0.2 kg/m² improvement in the twice-daily group was significantly greater than that seen in the four-times-daily group (95 percent confidence interval, 0.01 to 0.39).

TABLE 6
Within-group changes in body mass index during two
weeks of regular hypertonic saline inhalation. Means
(95 percent confidence intervals) are presented.

| | Twice Daily | Four Times Daily |
|---|---|---|
| Outcome | (N = 20) | (N = 20) |
| Absolute change in body | 0.20 (0.06 to 0.34) | 0.00 (−0.14 to 0.14) |
| mass index from baseline | | |
| (kg/m²) | | |

One subject commenced inhaled Tobramycin in the twice-daily group. Two subjects in the four-times-daily group commenced oral Ciprofloxacin. These between-group differences were non-significant on Fisher's Exact test.

The within-group improvements in oxygenation during the two-week treatment period for each group are shown in Table 7. The between-group comparisons were not significant: 0.5 absolute percent greater in the twice-daily group (95 percent confidence interval, −0.8 to 1.9).

TABLE 7
Within-group changes in oxygenation during two weeks
of regular hypertonic saline inhalation. Means (95
percent confidence intervals) are presented.

| | Twice Daily | Four Times Daily |
|---|---|---|
| Outcome | (N = 20) | (N = 20) |
| Absolute change in body | 0.9 (0.0 to 1.8) | 0.4 (−0.7 to 1.4) |
| mass index from baseline | | |
| (kg/m²) | | |

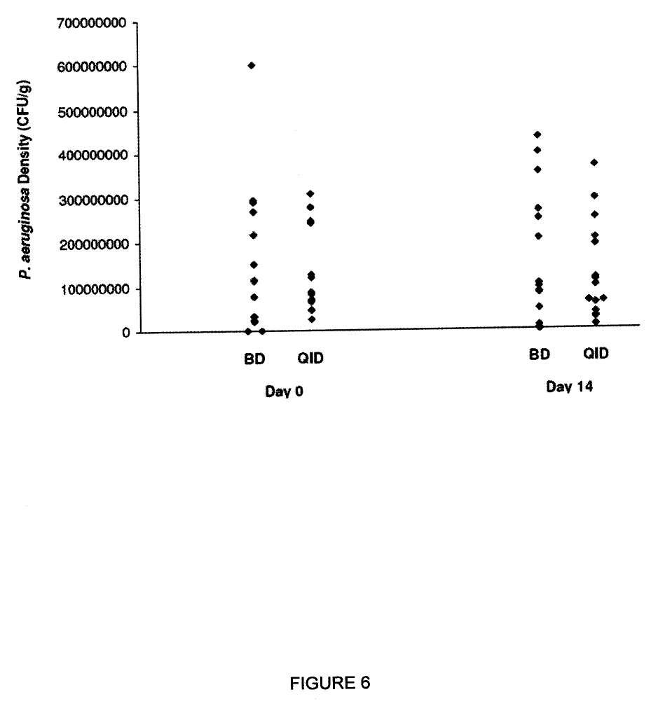

The density of P. aeruginosa in sputum is shown in FIG. 6. The change in density of P. aeruginosa in sputum from baseline was about 7×10⁶ (95 percent confidence interval, −70×10⁶ to 84×10⁶) in the twice-daily group and about 1×10⁶ (95 percent confidence interval, −41×10⁶ to 45×10⁶) in the four-times-daily group. The between-group difference in change from baseline was non-significant: about 6×10⁶ (95 percent confidence interval, −80×10⁶ to 91×10⁶).

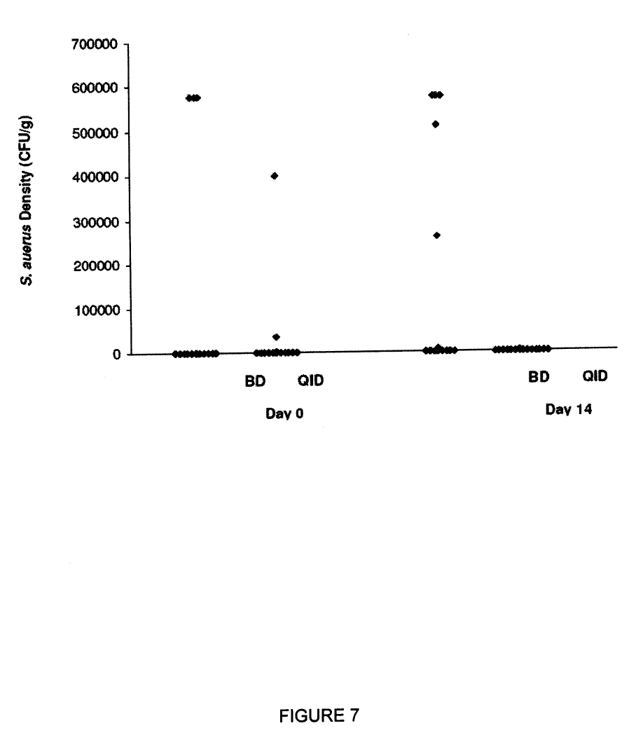

The density of S. aureus in sputum is shown in FIG. 7. The median (interquartile range) change in density of S. aureus in sputum from baseline was 0 (0 to 4000) in the twice-daily group and 0 (0 to 0) in the four-times-daily group. The between-group difference in change from baseline was non-significant (P=0.18).

There was one acquisition of P. aeruginosa in the twice-daily group and none in the four-times daily group during the two-week treatment period. There were two losses of S. aureus in each group during the treatment period.

The most common adverse events were increased cough and pharyngitis (Table 8). One case of increased cough persisted throughout the treatment period, although the subject stated that she found the coughing beneficial and it would not stop her from continuing to use the medication. One case of throat soreness persisted throughout the treatment period and resolved shortly thereafter. The remaining adverse events resolved within a maximum of five days, with four requiring a temporary cessation of dosing. The between-group difference in the total number of adverse reactions was not significant with an incidence rate ratio of 1.375 (95 percent confidence interval, 0.553 to 3.418). Restricting this analysis to only those events assessed by the subject to be related to the inhalation regimen, there were 7 in the twice-daily and 6 in the four-times-daily groups. The incidence rate ratio was again non-significant: 1.167 (0.392 to 3.471).

TABLE 8
Adverse events.

| | Twice | Four Times |
|---|---|---|
| Reaction | Daily | Daily |
| Increased cough | 4 | 3 |
| Pharyngitis | 2 | 1 |
| Chest tightness | 0 | 1 |
| Chest wall pain associated with coughing | 1 | 1 |
| Head cold | 1 | 1 |
| Vomiting not associated with coughing | 2 | 0 |
| Reflux associated with coughing | 1 | 0 |
| Haemoptysis | 0 | 1 |
| Total | 11 | 8 |

Returned sachets indicated mean (standard deviation) adherence of 91 (14) percent in the twice-daily group, but only 76 (19) percent in the four-times-daily group. Overall, 90% of subjects said they would use the therapy on a long-term basis, but two in the twice-daily group and 13 in the four-times-daily group would only do so if the dosing frequency were reduced.

Hypertonic saline given rapidly via the eFlow® Rapid nebulizer was well tolerated as a single dose. The about 106 mL rise in FEV₁ after bronchodilator and the subsequent about 131 mL fall after the first dose of hypertonic saline meant that subjects generally returned close to their original baseline. Overall there was a minimal acute detrimental effect on lung function which equated to a one percent relative change at the end of the tolerance test. This is the same as the overall acute change observed with slower delivery of hypertonic saline in the tolerance testing for the long-term trial.

Three (7%) of the 40 subjects had a acute fall in their FEV₁ of greater than about 15 percent in the tolerance test. This is slightly higher than the corresponding proportion in the hypertonic-saline group in the long-term trial: 2 (2%) of 83 subjects. The falls in FEV₁ after fast delivery were all temporary, however, with all three subjects having a greater FEV₁ than at baseline by 15 minutes later. One of the two subjects with an excessive acute fall after slow delivery had not recovered it at the 15 minute time point.

The acute changes in symptom severity with the initial dose were brief and acceptable to all subjects. The median rises on the 100-mm visual analogue scale in severity of cough (17 mm), dyspnoea (6 mm), wheeze (5 mm) and chest tightness (2 mm) were slightly larger than those that had been observed with slower delivery of hypertonic saline in the tolerance testing for the long-term trial: 4 mm, −1 mm, 1 mm and 4 mm, respectively. However, the rises in symptoms provoked by rapid delivery improved rapidly, with all four symptoms returning to below baseline levels within fifteen minutes of completing the inhalation.

Tolerance of twice-daily inhalations of hypertonic saline by the eFlow Rapid nebulizer was excellent, with all subjects completing the two-week treatment regimen and 90 percent willing to continue the regimen after the study. In contrast, the proportion of subjects who found the four-times-daily regimen intolerable exceeded our a priori limit of 15 percent.

Adherence was excellent in the twice-daily group. It equated with that seen in the first two-weeks of the long-term trial. Overall, tolerance to a single dose with fast delivery was acceptable, in terms of both symptom and FEV₁ responses. Continuing these inhalations on a twice-daily basis was also well tolerated. Increasing to a four-times-daily regimen did not significantly improve outcomes and reduced tolerability for a substantial proportion of subjects. Rapid delivery of hypertonic saline appeared to be a viable intervention for a long-term trial to investigate whether adherence and clinical outcomes can be improved over those achieved with slower delivery.

What is claimed is:

1. A system for administering hypertonic saline therapy to a patient afflicted with a condition, said system comprising:
at least one ampoule containing a sterile, preservative free, non-pyrogenic hypertonic saline solution having a concentration in the range of about 3.5% to about 9%.

2. The system of claim 1, wherein the hypertonic saline solution has a concentration of about 7%.

3. The system of claim 1, wherein the patient is afflicted with cystic fibrosis.

4. The system of claim 1, wherein said system further comprises a vibrating mesh nebulizer.

5. The system of claim 1, wherein said system further comprises a jet nebulizer.

6. The system of claim 1, wherein the therapy includes administering about a 7% hypertonic saline solution to a patient at least twice daily.

7. The system of claim 1, wherein the ampoule is filled with about 4 ml of a hypertonic saline solution.

8. The system of claim 1, wherein the hypertonic solution further includes an additional pharmacologically active agent.

9. The system of claim 8, wherein the pharmacologically active agent is one or more compounds selected from the group consisting of an anti-infective, an anti-mucolytic, a buffer, and an anti-inflammatory.
